(19) United States
(12) Patent Application Publication
(10) Pub. No.: US 2026/0166157 A1
(43) Pub. Date: Jun. 18, 2026

(54) SUSTAINED-RELEASE INJECTABLE PREPARATION COMPRISING DEXAMETHASONE ACETATE AND PREPARATION METHOD THEREFOR

(71) Applicant: G2GBIO, INC., Cheongju-si (KR)

(72) Inventors: Geonho KIM, Cheongju-si (KR); Sungwoong MOON, Daejeon (KR); Jinwoo LEE, Sejong-si (KR); Eunyoung SEOL, Daejeon (KR); Heeyong LEE, Daejeon (KR)

(21) Appl. No.: 19/125,368

(22) PCT Filed: Nov. 7, 2023
(86) PCT No.: PCT/KR2023/017791

§ 371 Date: Apr. 29, 2025

(30) Foreign Application Priority Data
Nov. 7, 2022 (KR) 10-2022-0147425
May 8, 2023 (KR) 10-2023-0059472

(51) Int. Cl. A61K 47/38; A61K 9/00; A61K 9/10; A61K 9/16; A61K 9/48

(52) U.S. Cl. CPC A61K 47/38; A61K 9/0019; A61K 9/0024; A61K 9/10; A61K 9/1635; A61K 9/1647; A61K 9/4858

(57) ABSTRACT

The present invention relates to a sustained-release injectable preparation containing dexamethasone acetate and a method for producing the same.

### TECHNICAL FIELD

The present invention relates to a long-acting microsphere preparations containing dexamethasone acetate, a method for producing the same, and a use of the microsphere preparations

### RELATED ART

Corticosteroids are a type of steroid hormone produced and secreted by the adrenal gland in response to pituitary adrenocorticotropic hormone, and are regulated by hypothalamic corticotropin-releasing hormone. This hormone is known to play a role in regulating major endocrine functions, including stress management and homeostasis regulation. Currently, corticosteroid drugs are used to treat various neurological diseases, inflammation, pain, autoimmune disorders, cancers and the like.

However, long-term use and high-dose use of steroid drugs can cause side effects and drug resistance, which can lead to a decrease in efficacy. In particular, long-term administration of high-dose corticosteroids can increase the patient's exposure to steroids, thereby causing various side effects. The interdependent mechanism among the hypothalamus which is responsible for secreting corticotropin-releasing factor, the pituitary gland which is responsible for secreting adrenocorticotropic hormone, and the adrenal cortex secreting cortisol, can be suppressed by the administration of corticosteroids. Therefore, there is a need for the development of sustained-release microsphere formulations of corticosteroids using drug delivery technology for treatments that require local administration of corticosteroid for a long period, such as treatment of inflammation.

Systemic administration of glucocorticoid can be used alone or in addition to topical glucocorticoids for the treatment of uveitis. However, the long-term exposure to high plasma concentrations (1 mg/kg/day for 2-3 weeks) of steroids is often required to achieve therapeutic levels in the eye.

Unfortunately, the high plasma levels of drug usually result in systemic side effects, such as hypertension, hyperglycemia, increased susceptibility to infection, gastric ulcers, psychosis, and other complications. In addition, topical, systemic, and periocular treatments of glucocorticoid must be closely monitored because of the long-term side effects associated with toxicity and chronic sequelae of systemic drug exposure.

### DETAILED DESCRIPTION

### Problem to be Solved

The present invention is attained to solve the problem of side effects caused by high systemic exposure of conventional corticosteroids, and the purpose of the present invention is to provide a sustained-release microsphere preparations containing dexamethasone acetate, which can maintain a therapeutic concentration range at the site of administration for a long period of time, and a method for manufacturing the same.

Another object of the present invention is to provide a sustained-release microsphere preparation containing dexamethasone acetate, which can maintain a therapeutic concentration range at the site of administration for a long period of time, while maintaining a very low plasma concentration of drugs, for example dexamethasone throughout the body of the subject being administered.

Another object of the present invention is to provide a medical or pharmaceutical use of the sustained-release microsphere preparations containing dexamethasone acetate, and more specifically, to provide a medical or pharmaceutical use of dexamethasone, for example, a use for treating a locally-occurring neurological disease, inflammation, pain, autoimmune disorder, cancers, arthritis, Meniere's disease, or macular degeneration.

### Means to Solve Problems

Hereinafter, the present invention will be described in more detail.

An embodiment of the present invention relates to a sustained-release injectable preparations including microspheres which contain dexamethasone acetate as an active ingredient and a biocompatible polymer, wherein the microspheres have 15 to 70 wt % of a content of the active ingredient, and an average diameter of 10 to 100 micrometers.

The sustained-release injectable preparations of dexamethasone according to the present invention may comprise one type of drug microsphere, or may comprise two or more different types of drug microspheres. For example, the sustained-release microspheres of dexamethasone according to the present invention may comprise two or more types of drug microspheres wherein are different in at least one selected from a group consisting of different compositions and manufacturing conditions. When the two or more types of drug microspheres are included, effects such as controlling the release period of the drug can be achieved.

A mixture of drug microspheres can be manufactured by the microsphere manufacturing method of steps (a) to (d). The different compositions and manufacturing conditions of the microspheres may be at least one selected from the group consisting of drug type, drug amount, polymer type, particle size distribution (e.g., average diameter), circularity, polymer amount, dispersed phase solvent, co-solvent type, co-solvent amount, continuous phase type, continuous phase amount, solidification temperature, solidification time, and theoretical content of drug, but are not limited thereto. The mixture may be obtained, for example, by mixing different drug microspheres having at least one difference selected from the group consisting of different compositions and manufacturing conditions, at a specific ratio.

As a specific example, the different drug microspheres may be drug microspheres having different components and composition ratios (hereinafter, drug microspheres having different compositions) and/or drug microspheres having different drug release properties, and for example, the different drug microspheres may have at least one difference selected from the group consisting of polymer type, polymer content, drug content, and the like of the microspheres. The different types of polymers in the microspheres may be at least one selected from the group consisting of polymers having different repeating units, polymers having the same repeating unit but different terminal groups, and polymers having different intrinsic viscosities.

The sustained-release injectable preparations of dexamethasone according to the present invention may be applicable to all medical or pharmaceutical uses of dexamethasone, and may be used, for example, for the treatment of locally-occurring neurological diseases, inflammation, pain, autoimmune disorders or tumors. Specifically, the preparations may be used for arthritis, Meniere's disease, macular degeneration or solid cancers.

Hereinafter, the present invention will be described in more detail.

A specific aspect of the present invention may include microparticles containing dexamethasone acetate as an active ingredient and a biodegradable polymer.

The active ingredient may be dexamethasone acetate, and may be at least one selected from the group consisting of dexamethasone 17-acetate and dexamethasone 21-acetate, and preferably may be dexamethasone 21-acetate having the following chemical formula 1.

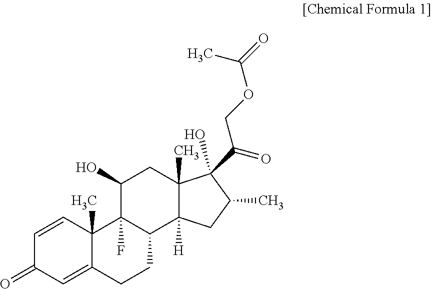

The content of the active ingredient (dexamethasone acetate) is 15 to 70 wt % based on 100 wt % of the total microspheres, for example, 15 wt % or more, 16 wt % or more, 17 wt % or more, 18 wt % or more, 19 wt % or more, 20 wt % or more, 25 wt % or more, 30 wt % or more, 35 wt % or more, 37 wt % or more, 38 wt % or more, 40 wt % or more, 41 wt % or more, 42 wt % or more, 43 wt % or more, 44 wt % or more, 45 wt % or more, 46 wt % or more, 47 wt % or more, 48 wt % or more, 49 wt % or more, 50 wt % or more, 51 wt % or more, 52 wt % or more, 53 wt % or more, 54 wt % or more, 55 wt % or more, 56 wt % or more, 57 wt % or more, 58 wt % or more, 59 wt % or more, 60 wt % or more, 65 wt % or more, or 67 wt % or more as a lower limit of content, and may be 70 wt % or less, 69 wt % or less, 68 wt % or less, 67 wt % or less, 66 wt % or less, 65 wt % or less, 64 wt % or less, 63 wt % or less, 62 wt % or less, 61 wt % or less, 60 wt % or less, 59 wt % or less, 58 wt % or less, 57 wt % or less, 56 wt % or less, 55 wt % or less, 54 wt % or less, 53 wt % or less, 52 wt % or less, 51 wt % or less, or 50 wt % or less as a upper limit of content, or may be a numerical range formed by a combination of the upper and lower limits.

The drug microspheres containing dexamethasone acetate according to the present invention have a uniform particle distribution, and the microspheres containing dexamethasone acetate have a smaller deviation during injection and can be administered in a more accurate amount compared to microspheres having non-uniform particle distribution. It is preferable that the span value of the size distribution (particle size distribution) of the microspheres containing dexamethasone acetate of the present invention is less than 1.1, 1.05 or less, or 1.0 or less. Specifically, the span value can be calculated according to the following mathematical formula 1.

$$Span value of particle diameter = ( D {v}_{0.9} - D {v}_{0.1} ) / {Dv}_{0.5} \qquad [ Mathematical Formula 1 ]$$

The terms “size distribution”, “span value” or “span value of particle size” used in the present invention are referred to indices representing the uniformity of particle size of microspheres, and mean a value obtained by the mathematical formula of particle size distribution (span value)=(Dv0.9-Dv0.1)/Dv0.5. Here, Dv0.1 means a particle size corresponding to 10% of % by volume in the particle size distribution curve of microspheres, Dv0.5 means a particle size corresponding to 50% of % by volume in the particle size distribution curve of microspheres, and Dv0.9 means a particle size corresponding to 90% of % by volume in the particle size distribution curve of microspheres. The span value of the particle diameter can be analyzed by measuring the particle size by injecting a sample solution containing microspheres into a particle size analyzer, but is not limited thereto.

The average circularity of the drug microspheres containing dexamethasone acetate according to the present invention may be 0.87 to 1.00, and the circularity span value representing the circularity distribution may be 0.01 to 0.05.

The circularity is sometimes described in the literature as the difference between the shape of a particle and a perfect sphere. Value range of the circularity is from 0 to 1, where a circularity of 1 represents a perfect spherical particle or disk particle as measured in a two-dimensional image. Circularity can be obtained from the following Mathematical formula, where P represents the perimeter length of the particle and A represents the projected area of the particle (the two-dimensional descriptor).

$$C = \sqrt{\frac{4 π A}{{P}^{2}}} \qquad [ Mathematical Formula 2 ]$$

The average circularity of microspheres may be 0.87 to 1.00, 0.88 to 1.00, 0.089 to 1.00, 0.90 to 1.00, 0.91 to 1.00, 0.87 to 0.99, 0.88 to 0.99, 0.089 to 0.99, 0.90 to 0.99, 0.91 to 0.99, 0.87 to 0.98, 0.88 to 0.98, 0.089 to 0.98, 0.90 to 0.98, 0.91 to 0.98, 0.87 to 0.97, 0.88 to 0.97, 0.089 to 0.97, 0.90 to 0.97, 0.91 to 0.97, 0.87 to 0.96, 0.88 to 0.96, 0.089 to 0.96, 0.90 to 0.96, 0.91 to 0.96, 0.87 to 0.95, 0.88 to 0.95, 0.089 to 0.95, 0.90 to 0.95, or 0.91 to 0.95.

Herein, the average circularity of particles can be analyzed using a Particle Image Analysis System, and the distribution degree of circularity of microparticles can be numerically represented. The average circularity and circularity span value can also be calculated accordingly. When the circularity of microparticles according to the present invention increases, it leads to a decrease in the roughness and surface area of the microparticles, and can also lower the crystallinity of the drug inside the microparticles. This has a technical significance in that it affects the release pattern.

The microparticles of the present invention may have less than 0.05, for example, 0.049 or less, 0.045 or less, 0.043 or less, 0.042 or less, 0.041 or less, 0.040 or less, 0.039 or less, or 0.038 or less of a circularity span value of particles as represented by the following mathematical formula 3. The circularity refers to the degree of circularity of microspheres containing dexamethasone acetate according to the present invention, and the circularity span value can be obtained by the following mathematical formula.

$$Circularity span value = ( C 90 - C 10 ) / C 50 \qquad [ Mathematical Formula 3 ]$$

Here, C90 means the area corresponding to 90% to 100% of circularity in the cumulative distribution curve of particle circularity (horizontal axis is particle circularity and vertical axis is percentage of particle, %), C50 means the area corresponding to 50% to 100% of circularity in the circularity distribution curve, and C10 means the area corresponding to 10% to 100% of circularity in the circularity distribution curve.

The average particle diameter of the drug microparticles according to the present invention may be about 10 to 100 μm, higher than 10 μm to 100 μm or less, 11 to 100 μm, 12 to 100 μm, 15 to 100 μm, 20 to 100 μm, 25 to 100 μm, 30 to 100 μm, about 10 to 95 μm, higher than 10 μm to 95 μm or less, 11 to 95 μm, 12 to 95 μm, 15 to 95 μm, 20 to 95 μm, 25 to 95 μm, 30 to 95 μm, about 10 to 90 μm, higher than 10 μm to 90 μm or less, 11 to 90 μm, 12 to 90 μm, 15 to 90 μm, 20 to 90 μm, 25 to 90 μm, 30 to 90 μm, about 10 to 85 μm, higher than 10 μm to 85 μm or less, 11 to 85 μm, 12 to 85 μm, 15 to 85 μm, 20 to 85 μm, 25 to 85 μm, or 30 to 85 μm, for example about 30 to 50 μm, but not limited thereto. The term “average particle size” or “average particle diameter” used in the present invention refers to the particle size corresponding to 50% of % by volume in the particle size distribution curve, which means the average particle diameter (median diameter) and is represented as D50 or D(v, 0.5).

The drug microspheres containing dexamethasone acetate according to the present invention include pores of a certain size, and specifically, may have at least one pore properties selected from the group consisting of a porosity of the drug microspheres of 8% or less, a maximum diameter of the pores in the drug microspheres of 8 micrometers (μm) or less, and an average diameter of the pores within the drug microspheres of 0.3 micrometers (μm) or less.

Specifically, the porosity of the drug microspheres containing dexamethasone acetate according to the present invention may be 8% or less, 7.5% or less, 7% or less, 6.5% or less, 6% or less, 5.5% or less, or 5% or less. The more internal pores there are, the less likely it is that sustained release will occur after administration of the microspheres, and a drug burst may occur during the release period. Therefore, the fewer internal pores there are, the more stable the release can be.

The maximum size of the pores in the drug microspheres containing dexamethasone acetate according to the present invention may be 8 micrometers (μm) or less, 7 micrometers (μm) or less, 6 micrometers (μm) or less, 5 micrometers (μm) or less, 4 micrometers (μm) or less, 3 micrometers (μm) or less, 2 micrometers (μm) or less, 1 micrometer (μm) or less, or 0.5 micrometer (μm) or less, and may be, for example, 0.01 to 8 micrometers (μm).

The drug microspheres containing dexamethasone acetate according to the present invention include pores of a certain size, and specifically, the average diameter of the pores in the drug microspheres may be 0.3 micrometers (μm) or less, 0.25 micrometers (μm) or less, 0.2 micrometers (μm) or less, or 0.15 micrometers (μm) or less, for example, 0.01 to 0.3 micrometers (μm).

The release characteristics of the microspheres according to the present invention can have a characteristic of stable long-term release of drug with a low release amount for 24 hours (1 day) from the time of drug administration. For example, in an in vitro drug release test using a phosphate buffer (pH 7.4), the released amount of drug for 24 hours can be 15% or less, 14% or less, 13.5% or less, 10% or less, 9% or less, 8% or less, 7% or less, 6% or less, 5% or less, or 4.5% or less, based on 100% of the drug contained in the microspheres.

In the in vivo drug release experiment of the drug microspheres using SD rats, the cumulative released amount of drug, for example dexamethasone for 24 hours may be 15% or less, 14% or less, 13.5% or less, 10% or less, 9% or less, 8% or less, 7% or less, 6% or less, 5% or less, or 4.5% or less, based on 100% of the drug contained in the microspheres. The amount of released drug is a measurement of the drug concentration in the blood of the experimental animal, and the measured drug may be an analysis of the content of dexamethasone free base, or total contents of dexamethasone free base and dexamethasone acetate, and more specifically, may be dexamethasone free base.

Herein, the term “subject” or “individual” includes mammals, especially humans, and the administration plan, administration interval, dosage, etc. can be easily set, changed, and adjusted by those skilled in the art by the above-mentioned factors.

Specifically, the administration interval of the sustained-release preparation according to the present invention may vary depending on the intended use and purpose, and may be set to, for example, 1 week, 1 month, 3 months, or 6 months, but is not limited thereto.

The preparation relates to a sustained-release injectable preparation for local administration, which is intraarticular administration, subcutaneous administration, intradermal administration, intramuscular administration, intratumoral administration, intraocular administration, intravitreal administration, or intratympanic administration. The preparation relates to a sustained-release injectable preparation for local administration, which is for use in arthritis, Meniere's disease, macular degeneration, or solid tumor.

The microspheres contain a biocompatible polymer together with the active ingredient, and the biocompatible polymer applicable to the present invention includes, but is not limited to, a biodegradable polymer. The polymer may be a biodegradable polymer having an intrinsic viscosity of 0.16 to 1.9 dL/g, 0.10 to 1.3 dl/g, or preferably 0.16 dl/g to 0.75 dL/g, in consideration of the factors such as the release characteristics of the drug and the manufacturing process. The intrinsic viscosity refers to that measured at a concentration of 0.1% (w/v) in chloroform at 25° C. using an Ubbelohde viscometer.

The weight average molecular weight of the biocompatible polymer is not particularly limited, but its lower limit may be 5,000 or more or preferably 10,000 or more, and its upper limit may be 500,000 or less, or preferably 200,000 or less.

Specifically, the type of the biodegradable polymer is not particularly limited, but may be at least one selected from the group consisting of, for example, polyethylene glycol-poly(lactide-co-glycolide) block-copolymer, polyethylene glycol-polylactide block-copolymer, polyethylene glycol-polycaprolactone block-copolymer, polylactide, polyglycolide, poly(lactide-co-glycolide), poly(lactide-co-glycolide) glucose, polycaprolactone, and mixtures thereof, and particularly, polylactide, poly(lactide-co-glycolide) and polycaprolactone may be used.

When poly(lactide-co-glycolide) is used as the biodegradable polymer, the molar ratio of lactic acid to glycolic acid in the copolymer can be 99:1 to 50:50, and preferably 50:50, 75:25, or 85:15.

When two or more of the biodegradable polymers are included, the types of the polymers exemplified above may be a combination or blend of polymers that are different from each other, but may also be a combination of polymers of the same type having different intrinsic viscosities and/or monomer ratios (for example, a combination or blend of two or more poly(lactide-co-glycolide) having different intrinsic viscosities), or may be polymers of the same type having different terminal groups (for example, a terminal group is an ester or a terminal group is an acid).

Examples of commercially available biodegradable polymers that can be used in the present invention include either singly or in combination or blends of Evonik's Resomer series of RG502H, RG503H, RG504H, RG502, RG503, RG504, RG653H, RG752H, RG752S, 753H, 753S, RG755S, RG756S, RG858S, R202H, R203H, R205H, R202S, R203S, R205S; and Corbion's products of PDL 02A, PDL 02, PDL 04, PDL 05, PDLG 7502A, PDLG 7502, PDLG 7504A, PDLG 7504, PDLG 7507, PDLG 5002A, PDLG 5002, PDLG 5004A, PDLG 5004, PDLG 5010, PL 10, PL 18, PL 24, PL 32, PL 38, PDL 20, PDL 45, PC 02, PC 04, PC 12, PC 17, PC 24, etc. Those skilled in the art can appropriately select the appropriate molecular weight or blending ratio of the biodegradable polymer by considering the decomposition rate of the biodegradable polymer and the resulting drug release rate. In a specific embodiment, to prepare the microparticles according to the present invention, Resomer R755S (i.v.=0.50-0.70 dL/g; manufacturer: Evonik, Germany) or Resomer R752H (i.v.=0.16-0.24 dL/g; manufacturer: Evonik, Germany) can be used.

The method for manufacturing dexamethasone sustained-release microspheres according to the present invention can be performed by the O/W (oil in water) method, and specifically includes (a) a step of dissolving a biocompatible polymer and dexamethasone acetate in an organic solvent to prepare a dispersed phase, (b) a step of adding the dispersed phase prepared in step (a) to an aqueous solution phase (continuous phase) containing a surfactant to form an emulsion, (c) a step of extracting an organic solvent from the dispersed phase in the emulsion state formed in step (b) into a continuous phase and evaporating the organic solvent to form microspheres, and (d) a step of recovering the microspheres from the continuous phase of step (c) to produce the sustained-release microspheres containing dexamethasone.

The dexamethasone sustained-release microspheres according to the present invention may include two or more types of drug microspheres, at least one of which is different from the group consisting of different compositions and manufacturing conditions. When the two or more types of drug microspheres are included, effects such as controlling the release period of the drug can be achieved.

A mixture of the drug microspheres can be prepared by the method for preparing microspheres of steps (a) to (d). The different compositions and manufacturing conditions may be at least one selected from the group consisting of drug amount, polymer type, particle size distribution (e.g., average diameter), circularity, polymer amount, dispersed phase solvent, co-solvent type, co-solvent amount, continuous phase type, continuous phase amount, solidification temperature, solidification time, and theoretical content of drug, but are not limited thereto. The mixing may be, for example, mixing at a specific ratio at least two different drug microsphere selected from the group consisting of different compositions and preparation conditions. As a specific example, the two different drug microspheres may be drug microspheres having different components and composition ratios (hereinafter, drug microspheres having different compositions) and/or drug microspheres having different drug release characteristics, and for example, at least one difference selected from the group consisting of polymer type, polymer content, drug content, etc. of the microspheres. The different types of polymers in the microspheres may be at least one different type selected from the group consisting of a repeating unit of the polymer, a terminal group of the polymer, a molecular weight of the polymer, and an intrinsic viscosity of the polymer.

The method for manufacturing drug microspheres according to the present invention not only has a uniform particle size distribution, but also has a high microsphere production yield (%).

The microsphere production yield (w/w %) is the value obtained by dividing the weight of the obtained microspheres by the total weight including the polymer and the drug, and converting it into a percentage. The microsphere production yield (w/w %) of the method for manufacturing drug microspheres according to the present invention may be 50% or more. Specifically, the production yield in the present specification may be obtained according to the following Mathematical formula 4.

$$Yield ( \% ) = 100 * ( weight of the obtained microspheres / \\ weight of input raw material ( drug + polyme r ) ) \qquad [ Mathematical Formula 4 ]$$

In addition, the method for manufacturing drug microspheres according to the present invention not only has a high yield, but these microspheres may have a particle size span value of 1.2 or less, 1.1 or less, or 1.0 or less. Accordingly, the method for manufacturing drug microspheres of the present invention has a high production yield, while having excellent uniformity of particle size and a very low span value of the size distribution (particle diameter distribution) of microspheres containing dexamethasone acetate.

Accordingly, the drug microspheres may have one or more properties of:
1.2 or less of a diameter span value of the drug microspheres,
0.87 to 1.00 of an average circularity of the drug microspheres,
0.01 to 0.05 of a circularity span value of the drug microspheres,
8% or less of a porosity of the drug microspheres,
8 micrometer (μm) or less of a maximum diameter of pores in the drug microspheres, and
0.3 micrometer (μm) or less of an average diameter of pores in the drug microspheres.

The encapsulation efficiency of the method for manufacturing dexamethasone acetate microspheres according to the present invention can be 80% or more, 85% or more, or 89% or more. Specifically, it is an excellent method having a high encapsulation efficiency while including a high content of drug, since the encapsulation efficiency is very high while encapsulating 15 wt % or more of drug. Particularly, when the content of the drug encapsulated within the microspheres is 15 wt % or more, 20 wt % or more, 30 wt % or more, 35 wt % or more, 40 wt % or more, 45 wt % or more, 50 wt % or more, or 55 wt % or more, the method has an encapsulation efficiency of 89% or more, 90% or more, or 93% or more. The encapsulation efficiency of the drug encapsulated in the drug microspheres is represented as a percentage value by dividing % by weight of the encapsulated drug based on 100% of the total weight of the drug microspheres by % by weight of the drug based on 100% of the total weight of the drug and polymer used as raw materials.

A specific aspect of the present invention relates to a method for producing biodegradable microspheres, comprising:
(a) dissolving a biodegradable polymer and a drug in an organic solvent to form a dispersed phase solution;
(b) homogeneously mixing the biodegradable polymer solution prepared in step (a) with an aqueous solution containing a surfactant to form an emulsion including a dispersed phase solution and an aqueous solution containing the surfactant as a continuous phase;
(c) extracting an organic solvent from the dispersed phase of the emulsion prepared in step (b) toward the continuous phase and evaporating the organic solvent to prepare microspheres; and
(d) recovering the microspheres from the emulsion of step (c).

In the manufacturing method, dexamethasone acetate as the drug is as described above.

In step (c), a step including removing a portion of the continuous phase containing the extracted organic solvent and supplying a new continuous phase may be additionally included.

Optionally, in step (c), a solidification process of heating the temperature of the continuous phase for a predetermined period of time is additionally performed to modify the surface of the microspheres, thereby controlling the initial release of the drug from the sustained-release microspheres and/or efficiently removing the organic solvent. When the temperature is controlled by applying heat to the continuous phase, the temperature range may be controlled at a glass transition temperature (Tg) of the polymer used or higher, for example, within a range set with the glass transition temperature (Tg) of the polymer as a lower limit and (the glass transition temperature (Tg) of the polymer+30° C.) as a upper limit. In the step (a), the biocompatible polymer or the biodegradable polymer is as described above.

By using the property of the organic solvent not to be miscible with water in step (a), the drug and the biodegradable polymer solution can be homogeneously mixed in a continuous phase to form an emulsion in the step (b) described below. The type of the solvent for dissolving the drug and the biodegradable polymer is not particularly limited, but preferably, may be at least one selected from the group consisting of dichloromethane, chloroform, ethyl acetate, acetone, acetonitrile, dimethylformamide, methyl ethyl ketone, acetic acid, methyl alcohol, ethyl alcohol, propyl alcohol, benzyl alcohol or a mixed solvent thereof, or more preferably, dichloromethane and ethyl acetate. The amount of the organic solvent used may be such that the concentration of the polymer in the dispersed phase solution including the polymer is 5% to 30 wt % or less.

More specifically, in the step (a), when the organic solvent may be used with a co-solvent for example, at least one selected from the group consisting of benzyl alcohol (BnOH) and dimethylformamide (DMF), or preferably benzyl alcohol. The co-solvent is used to dissolve the drug, and has advantages of preparing uniform particles and also manufacturing with a high encapsulation efficiency and yield of microsphere production. The amount of the co-solvent may be 5% to 65 wt % based on 100 wt % of the total dispersed phase including the drug, the polymer, organic solvent, and the co-solvent. The co-solvent has advantages of helping to dissolve the drug, improving the uniformity of the particles, and manufacturing with a high drug encapsulation efficiency and microsphere production yield.

The amount of the co-solvent may be 5 to 65 wt % or less based on the total dispersed phase including the drug, the polymer, the organic solvent, and the co-solvent.

The manufacturing method of the present invention includes the step of (b) homogeneously mixing the drug and biodegradable polymer solution manufactured in step (a) into an aqueous solution containing a surfactant, thereby forming an emulsion containing the biodegradable polymer solution as a dispersed phase and the aqueous solution containing a surfactant.

The method of homogeneously mixing the biodegradable polymer solution and the aqueous solution containing the surfactant in the step (b) is not particularly limited, but may preferably be performed using a high-speed mixer, an inline mixer, a membrane emulsion method, a microfluidics emulsion method, a spray drying method, or the like. When forming an emulsion containing the biodegradable polymer solution and the aqueous solution containing the surfactant as in the step (b), the biodegradable polymer solution is homogeneously dispersed in the aqueous solution to form a dispersed phase in the form of droplets.

Therefore, the aqueous solution containing the surfactant as the continuous phase used in the step (b) has a property of not being miscible with the organic solvent in the biodegradable polymer solution or the dispersed phase.

The type of the surfactant used in the step (b) is not particularly limited, and any surfactant can be used, if it can help the biodegradable polymer solution as the continuous phase to form a stable droplet dispersion phase in the aqueous solution phase. The surfactant can be preferably selected from the group consisting of methylcellulose, polyvinylpyrrolidone, carboxymethylcellulose, lecithin, gelatin, polyvinyl alcohol, polyoxyethylene sorbitan fatty acid ester, and polyoxyethylene castor oil derivatives and mixtures thereof, or most preferably, polyvinyl alcohol can be used.

In the step (b), the content of the surfactant in the aqueous solution containing the surfactant may be 0.01% (w/v) to 20% (w/v), or preferably 0.1% (w/v) to 5% (w/v), based on the total volume of the aqueous solution containing the surfactant.

The method for homogeneously mixing the dispersed phase solution containing dexamethasone acetate and a biodegradable polymer with the continuous phase containing a surfactant in the step (b) is not particularly limited, but may be performed using a high-speed stirrer, an inline mixer, an ultrasonic disperser, a static mixer, a membrane emulsion method, a microfluidic emulsion method, a spray drying method, or the like. It is difficult to obtain a uniform emulsion, when forming an emulsion using a high-speed stirrer, an inline mixer, an ultrasonic disperser, or a static mixer, it is difficult to obtain a uniform emulsion. Accordingly, it is preferable to additionally perform a process of selecting the particle size between steps (c) and (d) described below.

In the step (b), the content of the surfactant in the continuous phase containing the surfactant may be 0.01 wt % to 20 wt %, or preferably 0.1 wt % to 5 wt %, based on the total volume of the continuous phase including the surfactant. When the content of the surfactant is less than 0.01 wt %, a dispersed phase or emulsion in the form of droplets in the continuous phase may not be formed, and when the content of the surfactant exceeds 20 wt %, it may be difficult to remove the surfactant due to an excessive amount of surfactant after the particles formation in the continuous phase.

The method for producing sustained-release microspheres of dexamethasone according to the present invention includes the steps of (c) extracting an organic solvent as a continuous phase from the dispersed phase in the emulsion state produced in step (b) and evaporating the organic solvent to form microspheres, and (d) recovering the microspheres from the continuous phase of step (c) to produce sustained-release microspheres containing dexamethasone.

In the step (c), when the emulsion including the dispersed phase in the form of droplets and the continuous phase containing the surfactant is maintained or stirred at a temperature below the boiling point of the organic solvent for a certain period of time, for example, 2 hours to 48 hours, 2.5 hours to 36 hours, 2.5 hours to 30 hours, 2.5 hours to 25 hours, for specifically 3 hours or 24 hours, the organic solvent can be extracted toward a continuous phase from the biocompatible polymer solution in which the dexamethasone in the form of droplets is dispersed. A portion of the organic solvent extracted toward a continuous phase can be evaporated from the surface of the emulsion. As the organic solvent is extracted and evaporated from the biocompatible polymer solution in which the dexamethasone in the form of droplets is dispersed, the dispersed phase in the form of droplets can be solidified to form microspheres.

In order to further efficiently remove the organic solvent in the step (c) and to control the drug release characteristics of the drug microspheres, a solidification process may be additionally performed in which the temperature of the continuous phase is heated to the boiling point of the organic solvent or higher, for a certain period of time. In a specific embodiment, in order to efficiently remove dichloromethane used in the production of microspheres according to the present invention, the continuous phase may be heated to a temperature of 45° C., which exceeds the boiling point of dichloromethane of 39.6° C., and maintained for 2 to 6 hours, for example, 3 hours.

In the present invention, by removing a portion of the continuous phase containing the organic solvent extracted from the dispersed phase in the step (c) and supplying a new aqueous solution containing a surfactant, which is capable of replacing the removed continuous phase, the organic solvent present in the dispersed phase is sufficiently extracted and evaporated into the continuous phase, thereby efficiently minimizing the residual amount of the organic solvent.

In the step (c), ethanol may be added to the continuous phase in order to further efficiently remove the organic.

In the step (d), the method of recovering the dexamethasone sustained-release microspheres can be performed using various known techniques, and for example, methods such as filtration or centrifugation can be used.

Between the steps (c) and (d), the remaining surfactant can be removed through filtration and washing, and the microspheres can be recovered by filtering again. The washing step for removing the remaining surfactant can be typically performed using water, and the washing step can be repeated several times.

In addition, as described above, when an emulsion is formed using a high-speed stirrer, an inline mixer, an ultrasonic homogenizer, or a static mixer in the step (b), a particle size selection process can be additionally performed between the steps (c) and (d) to obtain uniform microspheres. A sieving process can be performed using a known technique, and small and large particles of microspheres can be filtered out with using sieves having different sizes to obtain microspheres having uniform size.

The method for manufacturing dexamethasone sustained-release microspheres according to the present invention can obtain finally dried microspheres by drying the obtained microspheres using a conventional drying method after the step (d) or after the filtration and washing steps.

The dexamethasone sustained-release microspheres according to the present invention may be a mixture of different drug microspheres having at least difference selected from a group consisting of different compositions and manufacturing conditions. The mixture of the drug microspheres may be manufactured by the microsphere manufacturing method of steps (a) to (d). The different compositions and manufacturing conditions may be at least one selected from the group consisting of a type of drug, an amount of drug, a type of polymer, an amount of polymer, a dispersed phase solvent, a co-solvent type, an amount of co-solvent, a type of continuous phase, an amount of continuous phase, a solidification temperature, a solidification time, and a theoretical content of the drug, but are not limited thereto. The mixing may be, for example, a mixture of different drug microspheres having at least one difference selected from the group consisting of different compositions and manufacturing conditions at a specific ratio.

Specifically, the method for producing a composition of drug microspheres in which two or more different types of microspheres are mixed at a specific ratio according to the present invention may include a step of repeating the process of producing microspheres in steps (a) to (d) at least twice to produce two or more different type of microspheres, and (e) mixing two or more different types of microspheres at an appropriate ratio.

In another aspect, a method for producing a composition of drug microspheres in which two or more different microspheres according to the present invention are mixed at a specific ratio may include:
(a′) a step of dissolving a biodegradable polymer having different compositions and a drug in an organic solvent to form two or more dispersed phase solutions;
(b′) a step of homogeneously mixing the two or more dispersed phases produced in step (a′) into each aqueous solution containing a surfactant, thereby forming two or more emulsions including the dispersed phase solution and the aqueous solution containing the surfactant as a continuous phase;
(c′) a step of extracting the organic solvent from the dispersed phase in the emulsion produced in step (b′) toward the continuous phase and evaporating the organic solvent to produce microspheres; and
(d′) a step of recovering the microspheres from the emulsion of step (c′).

### Effects of the Invention

The present invention relates to a sustained-release injectable preparation comprising dexamethasone acetate and a method for preparing the same.

### BRIEF DESCRIPTION OF DRAWINGS

FIG. 1 shows the change in blood concentration of dexamethasone over time after administering drug microspheres of Examples 4 and 5 to rats.

FIG. 2 shows the change in blood concentration of dexamethasone over time after administering drug microspheres of Example 10 to rats.

FIG. 3 shows the change in blood concentration of dexamethasone over time after administering drug microspheres of Examples 23 to 25 to rats.

FIG. 4a to FIG. 4d show cross-sections of microspheres of Examples 5, 16, 23, and 26 observed using a scanning electron microscope (SEM).

FIG. 5a and FIG. 5b show cross-sections of microspheres of Examples 6 and 7 observed using a scanning electron microscope (SEM).

FIG. 6 shows the cross-section of the microspheres of Comparative Example 1 observed by a scanning electron microscope (SEM).

FIG. 7 shows the cross-sections of the microspheres of Examples 3, 10, 11, and 16.

FIG. 8 shows the cross-sections of the microspheres of Comparative Examples 1 and 2.

FIG. 9 shows the cumulative AUC over time after administering the drug microspheres of Example 19 to rats.

### MODE OF INVENTION

The present invention will be described in more detail with reference to the following examples; however, the scope of protection of the present invention is not limited to the following exemplary examples.

### Example 1: Preparation of Sustained-Release Microspheres of dexamethasone acetate

The dispersed phase was prepared by mixing 1.60 g of Purasorb PDLG 7502A (i.v. 0.16-0.24 dl/g; manufacturer: Purac, Netherlands) as a biocompatible polymer, and 0.40 g of dexamethasone 21-acetate (manufacturer: Pfizer, USA), 4.00 g of dichloromethane, and 2.5 g of benzyl alcohol (BnOH) as a co-solvent.

The dispersed phase was stirred for 30 minutes or more to ensure sufficient dissolution before use. The continuous phase used a 0.5% (w/v) polyvinyl alcohol (viscosity: 4.8-5.8 mPa s) aqueous solution. When necessary, dispersed phase was used by adding sodium chloride. The prepared dispersed phase was injected into the continuous phase to prepare a microsphere suspension. The microsphere suspension was placed in a preparation container and stirred at a speed of 200 rpm, and the temperature of the preparation container was maintained at 25° C. After the injection of the dispersed phase was completed, the organic solvent was removed while maintaining the temperature of the microsphere suspension at 45° C. for 3 hours. After the removal of the organic solvent, the temperature of the microsphere suspension was lowered to 25° C., and followed by filtration and washing three times with tertiary distilled water to remove the residual polyvinyl alcohol, to obtain microspheres. The microspheres obtained at this step were lyophilized to recover sustained-release microspheres containing dexamethasone acetate.

In addition, the drug microspheres according to Examples 2 to 27 shown in Table 1 below were manufactured using substantially the same manufacturing method as the above, except that the manufacturing conditions were different from those of Example 1.

Specifically, manufacturing method of Examples 2 to 27 being different from Example 1 was varying the theoretical drug content or changing the type of polymer to manufacture drug microspheres. In addition, in order to efficiently minimize the residual amount of the organic solvent present in the dispersed phase in Examples 1 to 27, addition of ethanol or the exchange of continuous phase was performed during the solidification process, and an additional process of heating for a certain period of time was performed for solidification “before/after continuous phase exchange”. Specifically, the solidification temperature and time were 45° C. for 3 hours. The solidification temperature and time of Example 7 were 15° C. for 24 hours, and the solidification temperature and time of Example 9 were 25° C. for 24 hours. The amount of the continuous phase used in Examples 2 and 9 was 1,500 mL as the same as in Example 1, and 2,000 mL of the continuous phase was used in Examples 3 to 6, Example 8, and Examples 10 to 27. The continuous phase of Example 7 used 5,500 mL.

In addition, the continuous phase solutions of Examples 6 and 7 were 0.5% PVA, and the continuous phase solutions of Examples 2 to 5, Example 8, and Examples 10 to 27 were 0.5% PVA with addition of 2.5% NaCl. The continuous phase solution of Example 9 was 0.5% PVA with addition of 2.5% NaCl and ethanol. In Table 1 below, when there are two or more types of polymers, the ratios described represent the weight ratio of each polymer type based on 100 wt % of the polymers.

Examples 28 and 29 used the compositions containing two different drug microspheres, specifically, the composition of Example 28 contained the drug microspheres of Example 27 and Example 13 in a weight ratio of 70:30 based on 100% by weight of the total drug microspheres, and the composition of Example 29 contained the drug microspheres of Example 19, Example 27, and Example 13 in a weight ratio of 70:20:10 based on 100% by weight of the total drug microspheres.

TABLE 1

| | | | | Amount of | | |
|---|---|---|---|---|---|---|
| | | | | used | | |
| | | | | dispersed | Co- | Amount of |
| | | Batch | Target | phase | solvent | use d co- |
| Category | Polymer type | size(g) | Loading(%) | solvent (g) | type | solvent(g) |
| Example 1 | 7502A | 2 | 20 | 4 | BnOH | 2.5 |
| Example 2 | 752H:753H = 33:67 | 2 | 35 | 6.5 | BnOH | 2.5 |
| Example 3 | 7502A:7504A = 50:50 | 1 | 40 | 3 | BnOH | 2.5 |
| Example 4 | 6504A:PDL04A = 50:50 | 1 | 40 | 3 | BnOH | 2.5 |
| Example 5 | 6504A:PDL04A = 67:33 | 1 | 40 | 3 | BnOH | 2.5 |
| Example 6 | PDL05 | 0.7 | 20 | 9.14 | DMF | 0.57 |
| Example 7 | PDL05 | 0.5 | 40 | 4.89 | DMF | 0.81 |
| Example 8 | R203H | 1 | 40 | 2.88 | BnOH | 2.5 |
| Example 9 | 7502A | 2 | 20 | 4 | BnOH | 2.5 |
| Example 10 | PDL02 | 1 | 40 | 3 | BnOH | 2.5 |
| Example 11 | PDL04 | 1 | 40 | 3 | BnOH | 2.5 |
| Example 12 | 7504A | 1 | 40 | 3 | BnOH | 2.5 |
| Example 13 | PDL02A:PDL04A = 50:50 | 1 | 40 | 3 | BnOH | 2.5 |
| Example 14 | PDL02A:PDL04A = 33:67 | 1 | 40 | 3 | BnOH | 2.5 |
| Example 15 | 7504A | 1 | 50 | 3.33 | BnOH | 3.12 |
| Example 16 | 7502A:7504A = 67:33 | 1 | 50 | 3.33 | BnOH | 3.12 |
| Example 17 | 7502A:PDL04A = 33:67 | 1 | 50 | 3.33 | BnOH | 3.12 |
| Example 18 | 7504A:PDL04A = 50:50 | 1 | 50 | 3.33 | BnOH | 3.12 |
| Example 19 | PDL04A | 1 | 55 | 4.5 | BnOH | 3.4 |
| Example 20 | PDL04A | 1 | 60 | 2.67 | BnOH | 3.74 |
| Example 21 | 7510 | 1 | 60 | 2.67 | BnOH | 3.74 |
| Example 22 | 8505A | 1 | 60 | 2.67 | BnOH | 3.74 |
| Example 23 | PDL 04 | 1 | 60 | 2.67 | BnOH | 3.74 |
| Example 24 | PDL 05 | 1 | 60 | 2.67 | BnOH | 3.74 |
| Example 25 | PDL 06 | 1 | 60 | 2.67 | BnOH | 3.74 |
| Example 26 | PDL04A | 1 | 70 | 2.7 | BnOH | 4.37 |
| Example 27 | PDL04A | 1 | 40 | 2.8 | BnOH | 0.25 |

### Comparative Example 1: Preparation of Sustained-Release Microspheres of dexamethasone

The dispersed phase solution was prepared by mixing 1.4 g of Resomer RG 752H (i.v.=0.14-0.22 dL/g; manufacturer: Evonik, Germany) as a biocompatible polymer, and 0.6 g of dexamethasone (free base) (manufacturer: Farmabios, Italy) in a mixture of 7.0 g of dichloromethane and 3.0 g of DMSO and dissolving them until transparent.

The continuous phase solution used 2,000 mL of a 1.0% (w/v) aqueous solution of polyvinyl alcohol (viscosity: 4.8-5.8 mPa s), and the prepared dispersed phase solution was injected into the continuous phase in the preparation vessel to prepare a microsphere suspension.

The microsphere suspension was placed in the preparation vessel and stirred at a speed of 200 rpm, and the temperature of the preparation vessel was maintained at 25° C. After the dispersion phase injection was completed, the organic solvent was removed while maintaining the temperature of microsphere suspension temperature at 45° C. for 3 hours. After the organic solvent was removed, the microsphere suspension was cooled to 25° C., and then filtered and washed three times with tertiary distilled water to remove the residual polyvinyl alcohol, to produce microspheres. The microspheres obtained at this stage were lyophilized to recover the final sustained-release microspheres containing dexamethasone.

### Comparative Example 2 3: Preparation of Sustained-Release Microspheres of dexamethasone acetate

In Comparative Example 2, the dispersed phase solution was prepared by dispersing 1.6 g of Purac 7504A (i.v.=0.38-0.48 dL/g; manufacturer: Evonik, Germany) as a biocompatible polymer, and 0.4 g of dexamethasone 21-acetate (manufacturer: Henan lihua, China) in a mixture of 3.0 g of dichloromethane and 1.3 g of DMSO, and dissolving them until transparent. In Comparative Example 2, the continuous phase solution was used with 1,500 mL of 0.5% (w/v) aqueous solution of polyvinyl alcohol (viscosity: 4.8-5.8 mPa s) being added by 2.5% NaCl. In Comparative Example 2, the continuous phase was connected to an emulsifying device equipped with a porous membrane having pores of a diameter of 40 μm, and then the prepared dispersed phase was injected into the porous membrane together with the continuous phase to produce a microparticle suspension.

In Comparative Example 3, the dispersed phase solution was prepared by dispersing 0.6 g of PDL02 (i.v.=0.16-0.24 dL/g; manufacturer: Evonik, Germany) as a biocompatible polymer, and 0.4 g of dexamethasone 21-acetate (manufacturer: Henan lihua, China) into a mixture of 5.4 g of dichloromethane and 2.5 g of DMSO and dissolving them until transparent. Comparative Example 3 used 3,600 mL of 0.5% (w/v) aqueous solution of polyvinyl alcohol (viscosity: 4.8-5.8 mPa s) as the continuous phase. In Comparative Example 3, the continuous phase was connected to an emulsifying device equipped with a porous membrane having pores of a diameter of 10 μm, and the prepared dispersed phase was injected into the porous membrane together with the continuous phase to prepare a microsphere suspension.

In Comparative Examples 2 and 3, the microsphere suspension was placed in a preparation vessel and stirred at a speed of 200 rpm, and the temperature of the preparation vessel was maintained at 25° C. After the dispersion phase injection was completed, the temperature of the microsphere suspension was maintained at 45° C. for 3 hours to remove the organic solvent. After the organic solvent was removed, the temperature of the microsphere suspension was lowered to 25° C., followed by filtration and washing with tertiary distilled water three times to remove the residual polyvinyl alcohol, to produce microspheres. The microspheres obtained at this step were lyophilized to recover the sustained-release microspheres containing dexamethasone acetate.

### Test Example 1. Analysis of Circularity of Microspheres

This experiment was conducted to quantitatively measure the mean circularity and uniformity of circularity of the manufactured microspheres. The experimental procedure was as follows.

5 mg of microspheres were dispersed in 0.2 mL of distilled water and then applied to a slide glass. The applied sample was placed on the stage of an optical microscope (Eclipse E100, manufacturer: Nikon, Japan). The circularity value of the microspheres was measured through an image analysis system (BT-1600, Bettersize Instrument Ltd, Dandong, China) connected to the microscope, and the mean circularity and circularity span values were calculated and analyzed. The results of the circularity analysis are shown in Table 2 below, and the circularity span values in Table 2 are values calculated using the following mathematical formula 3.

$$Circularity span = ( C 90 - C 10 ) / C 50 \qquad [ Mathematical Formula 3 ]$$

In the mathematical expression 3, C90 means an area having a circularity value of 90% or more in the distribution curve of the circularity of the microspheres, C50 means an area having a circularity value of 50% or more, and C10 means an area having a circularity value of 10% or more.

TABLE 2

| | | | | Mean | Circularity |
|---|---|---|---|---|---|
| Category | C10 | C50 | C90 | Circularity | span value |
| Example 1 | 0.911 | 0.925 | 0.934 | 0.941 | 0.025 |
| Example 2 | 0.922 | 0.932 | 0.943 | 0.932 | 0.023 |
| Example 3 | 0.921 | 0.933 | 0.943 | 0.932 | 0.024 |
| Example 4 | 0.915 | 0.931 | 0.942 | 0.93 | 0.029 |
| Example 5 | 0.918 | 0.931 | 0.94 | 0.93 | 0.024 |
| Example 6 | 0.919 | 0.929 | 0.939 | 0.931 | 0.022 |
| Example 7 | 0.923 | 0.942 | 0.957 | 0.94 | 0.036 |
| Example 8 | 0.921 | 0.931 | 0.937 | 0.913 | 0.017 |
| Example 9 | 0.906 | 0.913 | 0.918 | 0.911 | 0.013 |
| Example 10 | 0.914 | 0.928 | 0.944 | 0.927 | 0.032 |
| Example 11 | 0.919 | 0.931 | 0.943 | 0.931 | 0.026 |
| Example 12 | 0.918 | 0.934 | 0.951 | 0.933 | 0.035 |
| Example 15 | 0.917 | 0.931 | 0.951 | 0.933 | 0.036 |
| Example 16 | 0.923 | 0.935 | 0.947 | 0.935 | 0.026 |
| Example 17 | 0.915 | 0.933 | 0.95 | 0.933 | 0.038 |
| Example 18 | 0.92 | 0.936 | 0.952 | 0.938 | 0.034 |
| Example 20 | 0.925 | 0.939 | 0.953 | 0.939 | 0.03 |
| Example 21 | 0.922 | 0.932 | 0.952 | 0.935 | 0.032 |
| Example 22 | 0.914 | 0.932 | 0.945 | 0.93 | 0.033 |
| Example 23 | 0.92 | 0.932 | 0.946 | 0.932 | 0.028 |
| Example 24 | 0.913 | 0.931 | 0.946 | 0.931 | 0.035 |
| Example 25 | 0.923 | 0.938 | 0.951 | 0.937 | 0.03 |
| Example 26 | 0.924 | 0.935 | 0.947 | 0.936 | 0.025 |
| Comparative | 0.917 | 0.941 | 0.97 | 0.941 | 0.056 |
| Example 1 | | | | | |
| Comparative | 0.903 | 0.928 | 0.949 | 0.93 | 0.056 |
| Example 2 | | | | | |
| Comparative | 0.937 | 0.961 | 0.999 | 0.964 | 0.064 |
| Example 3 | | | | | |

As can be seen in Table 2 above, the microspheres of Examples have an average circularity of 0.91 or more and a circularity span value of less than 0.05, representing that uniform, nearly perfect spherical microspheres with a very narrow circularity distribution were manufactured.

### Test Example 2. Analysis of Drug Content

In order to measure the dexamethasone acetate content in the microspheres manufactured in the Examples and Comparative Examples, the microspheres amount which corresponded to 2 mg of dexamethasone acetate content as a theoretical content of drug were completely dissolved in DMSO, and 10 μL of the solution was injected into HPLC and measured at a detection wavelength of 254 nm. The column used in this measurement was Inertsil C18 5 μm, 4.6×150 mm, and the mobile phase used an aqueous acetonitrile solution at a concentration of 20% to 50% by using a concentration gradient elution method.

In the following Table, an amount (%) of used drug refers to % by weigh (wt %) of the drug based on 100% of the total weight of the drug and polymer used as raw materials, and the amount of encapsulated drug (%) refers to % by weight of the encapsulated drug based on 100% of the total weight of the drug microspheres manufactured. The encapsulation efficiency of the drug encapsulated in the drug microspheres below is expressed as a percentage value, which is the numerical value obtained by dividing % by weight of the encapsulated drug based on 100% of the total weight of the drug microspheres, by % by weight of the input drug based on 100% of the total weight of the drug and polymer used as raw materials. The results of the drug content analysis are shown in Table 3 below. The drug encapsulation efficiency exceeding 100% in Table 3 below is interpreted to be caused by loss of the input polymer during the manufacturing process.

TABLE 3

| | | Amount of | |
|---|---|---|---|
| | Amount of used | encapsulated drug | Drug |
| Category | drug(wt %) | (wt %) | efficiency(%) |
| Example 1 | 20 | 18.8 | 93.9 |
| Example 2 | 35 | 31.8 | 90.8 |
| Example 3 | 40 | 37.2 | 93 |
| Example 4 | 40 | 40.7 | 101.7 |
| Example 5 | 40 | 43.5 | 108.7 |
| Example 6 | 20 | 19.5 | 97.7 |
| Example 7 | 40 | 35.9 | 89.7 |
| Example 8 | 40 | 41.2 | 103 |
| Example 9 | 20 | 22.6 | 112.9 |
| Example 10 | 40 | 43.9 | 109.7 |
| Example 11 | 40 | 42.2 | 105.6 |
| Example 12 | 40 | 43.5 | 108.7 |
| Example 13 | 40 | 42 | 105 |
| Example 14 | 40 | 42.1 | 105.3 |
| Example 15 | 50 | 53 | 106 |
| Example 16 | 50 | 47.9 | 95.9 |
| Example 17 | 50 | 50 | 100 |
| Example 18 | 50 | 51.1 | 102.1 |
| Example 19 | 55 | 61.7 | 112.2 |
| Example 20 | 60 | 59.3 | 98.8 |
| Example 21 | 60 | 63.3 | 105.5 |
| Example 22 | 60 | 60.3 | 100.5 |
| Example 23 | 60 | 61.5 | 102.5 |
| Example 24 | 60 | 61.5 | 102.4 |
| Example 25 | 60 | 59 | 98.3 |
| Example 26 | 70 | 70 | 100 |
| Example 27 | 40 | 40.1 | 100.3 |
| Example 28 | 40 | 41.4 | 103.4 |
| Example 29 | 50.5 | 55.4 | 109.1 |
| Comparative | 20 | 7.9 | 46.9 |
| Example 1 | | | |
| Comparative | 20 | 18.6 | 93.1 |
| Example 2 | | | |
| Comparative | 40 | 30.2 | 75.6 |
| Example 3 | | | |

As can be seen in Table 3 above, the encapsulation efficiency of O/W microspheres containing dexamethasone acetate as an active ingredient, which were manufactured according to the manufacturing conditions of the Examples, was found to be high at about 80% or more, whereas the encapsulation efficiency of O/W microspheres containing dexamethasone as an active ingredient, as in Comparative Example 1, was found to be considerably low at 46.9%.

### Test Example 3: Measurement of Production Yield and Span Value of Average Diameter of Microspheres

For the drug microspheres manufactured according to the Examples and Comparative Examples, the microsphere production yield was calculated according to the following mathematical formula 4. Specifically, the microspheres obtained in each of the Examples and Comparative Examples were quantitatively measured by using the weight of the microparticles recovered after the completion of freeze-drying. The weight of the microspheres in the container was measured using a balance (OHAUS, USA), and then divided by the total amount of the drug and polymer used in the manufacturing, and then converted as a percentage.

$$Yield ( \% ) = 100 \times \{ ( amount of recovered microspheres ) / ( amount \\ of input raw materials ( drug + polymer ) ) \} \qquad [ Mathematical Formula 4 ]$$

The average particle size (particle diameter, average diameter) and distribution of the microparticles as the particle size analysis of the microspheres was quantitatively measured using the laser diffraction method. Specifically, the manufactured microparticles and ultrapure water containing a surfactant for each sample were mixed with a vortex mixer for 20 seconds, and then placed in an ultrasonic generator and dispersed to prepare a sample solution for analysis. The sample solution for analysis was injected into a particle size analyzer (Microtrac Bluewave, Japan) to measure the particle size. The span value as an indicator of particle size uniformity was obtained according to the following mathematical formula 1.

$$Span value of particle diameter = ( D {v}_{0.9} - D {v}_{0.1} ) / {Dv}_{0.5} \qquad [ Mathematical Formula 1 ]$$

The microsphere production yields and span values of particle diameter of Examples 2, 12, and 15 and Comparative Examples 1 to 3 are shown in Table 4.

TABLE 4

| | Production yield of | Span value of particle | |
|---|---|---|---|
| Category | microspheres (w/w %) | diameter(D50) | |
| Example 2 | 67.5 | 0.65 | (36.7) |
| Example 12 | 73 | 0.99 | (42.3) |
| Example 15 | 68 | 0.82 | (41.1) |
| Comparative | 70 | 1.61 | (50.4) |
| Example 1 | | | |
| Comparative | 49.5 | 1.43 | (75.3) |
| Example 2 | | | |
| Comparative | 68 | 1.10 | (29.87) |

Example 3

### Test Example 4: Release Profile Characteristics of Drug (in-vitro Release Test)

This experiment was conducted in vitro to evaluate the initial drug release capability for sustained-release microspheres containing dexamethasone acetate. The experimental procedure is as follows.

Microsphere amount corresponding to 2 mg of dexamethasone acetate as theoretical amount and phosphate buffer (pH 7.4) were placed in a 50 mL conical tube and stored in a 37° C. incubator. 1 mL of solution was taken from the conical tube at predetermined time intervals and was supplemented with the same amount of phosphate buffer. The taken solution was filtered through a 0.45 μm syringe filter, and 40 μL was injected into HPLC. At this time, the HPLC column and operating conditions were the same as the HPLC analysis conditions of Example 2. The initial drug release of the microspheres manufactured in Examples 1 to 3 and Examples 10 to 27 was evaluated based on the HPLC analysis results.

TABLE 5

| | Amount of drug released |
|---|---|
| Category | for 24 hours (%) |
| Example 1 | 2.9 |
| Example 2 | 0.4 |
| Example 3 | 0.18 |
| Example 10 | 1.41 |
| Example 11 | 0.38 |
| Example 12 | 0.94 |
| Example 15 | 0.98 |
| Example 16 | 3.04 |
| Example 17 | 0.99 |
| Example 18 | 1.24 |
| Example 19 | 0.38 |
| Example 20 | 1.1 |
| Example 21 | 0.63 |
| Example 22 | 1.13 |
| Example 23 | 2.04 |
| Example 24 | 1.18 |
| Example 25 | 0.75 |
| Example 26 | 2.5 |
| Example 27 | 2.12 |

As can be seen in Table 5 above, the amount of drug released for 24 hours in vitro for the drug microspheres according to the Examples was evaluated to be 5% or less.

### Test Example 5. In-vivo Pharmacokinetic Test Using Rats

In order to evaluate the pharmacokinetics of the injectable preparation of sustained-release microspheres containing dexamethasone acetate manufactured in the above Examples, the blood dexamethasone concentration was measured over time after administration to rats.

The drug microspheres were suspended at an amount of drug microspheres corresponding to a dose of 0.3 mg/head as dexamethasone acetate in the microspheres manufactured with TL (target loading) of 35% to 55%, and then were injected subcutaneously into SD rats. Blood was collected every hour, and the blood dexamethasone concentration was measured using LC-MS/MS.

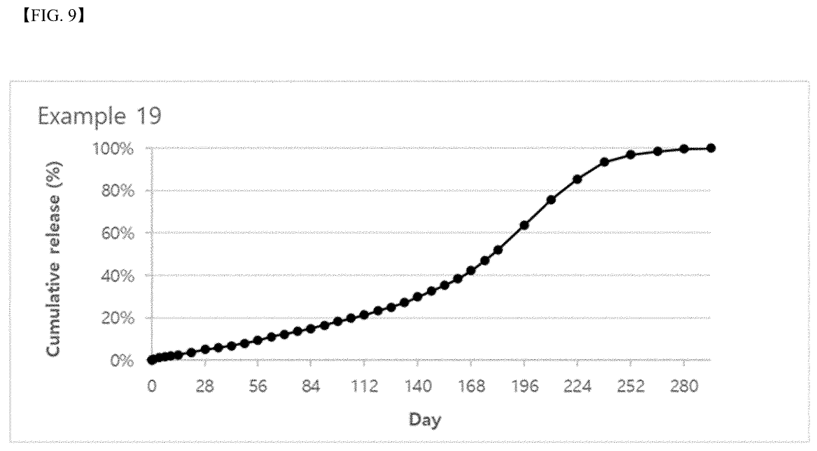

Specifically, the dexamethasone microspheres according to Examples 1, 2, 13, 14, and 19 were administered to rats, and the changes in the blood concentration of dexamethasone (free base) over time were measured and shown in Table 6. The dexamethasone microspheres according to Example 19 were administered to rats, and the cumulative AUC was obtained and shown in FIG. 9. Table 6 below shows the cumulative AUC (%) values over time, where h in the elapsed time represents hour, d represents day, and (nd) represents no measured value due to the end of the test period.

TABLE 6

| | | Exam- | Exam- | Exam- | Exam- | Exam- |
|---|---|---|---|---|---|---|
| Elapsed time | | ple 1 | ple 2 | ple 13 | ple 14 | ple 19 |
| 1 | h | 0 | 0 | 0 | 0 | 0 |
| 24 | h | 2.9 | 0.4 | 0.2 | 0.3 | 0.5 |
| 7 | d | 33.3 | 3.4 | 1.2 | 1.6 | 1.7 |
| 28 | d | 100 | 53.8 | 5.3 | 5.9 | 5.1 |
| 56 | d | nd | 100 | 11.1 | 13.3 | 9.5 |
| 84 | d | nd | nd | 19.7 | 30.2 | 15.2 |
| 112 | d | nd | nd | 46.7 | 83.2 | 21.6 |
| 140 | d | nd | nd | 89.2 | 98.3 | 30.3 |
| 168 | d | nd | nd | 99.2 | 100 | 42.7 |
| 196 | d | nd | nd | 100 | nd | 64.7 |
| 224 | d | nd | nd | nd | nd | 86.4 |
| 294 | d | nd | nd | nd | nd | 100 |

The experimental results of Examples 4 and 5 performed to evaluate the release profiles according to the drug content and polymer, represented that the polymer had a greater effect on the PK profile than the drug content.

The drug microspheres were suspended at an amount of drug microspheres corresponding to a dose of 0.3 mg/head as dexamethasone acetate in the microspheres manufactured with theoretical drug content of 40%, and then were injected subcutaneously into SD rats. Blood was collected every hour, and the blood dexamethasone concentration was measured using LC-MS/MS.

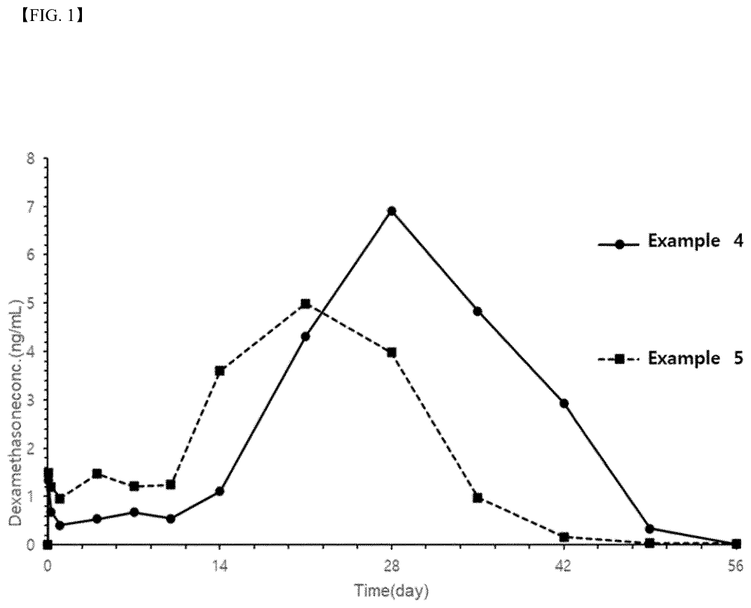

Specifically, the dexamethasone microspheres according to Examples 4 and 5 were administered to rats, and the changes in the blood concentration of dexamethasone (free base) over time were measured and shown in Table 7 and FIG. 1. Table 7 below shows the cumulative AUC (%) values over time, where h in the elapsed time represents hour and d represents day.

TABLE 7

| | | |
|---|---|---|
| elapsed time | Example 4 | Example 5 |

| | | | |
|---|---|---|---|
| 1 | h | 0 | 0 |
| 24 | h | 0.4 | 1.1 |
| 7 | d | 2.7 | 8.6 |
| 28 | d | 46.9 | 81.4 |
| 56 | d | 100 | 100 |

Referring to the experimental results of Examples 4 and 5 in Table 7, the drug release was completed on day 56, and the drug microspheres of Example 4 had a cumulative release rate of about 47% on day 28, which was a desirable release profile for one-month formulation. The experimental results of Examples 4 and 5 performed to evaluate the release profiles according to the drug content and polymer, represented that the polymer had a greater effect on the PK profile than the drug content.

In Addition, the drug microspheres of Example 10 were suspended at an amount of drug microspheres corresponding to a dose of 0.06 mg/head as dexamethasone acetate in the microspheres, and then were injected subcutaneously into SD rats. Blood was collected every hour, and the blood dexamethasone concentration was measured using LC-MS/MS.

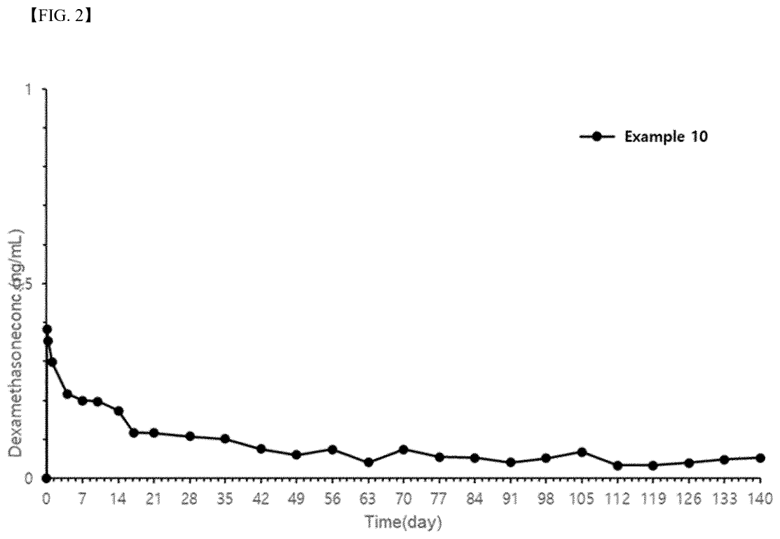

Specifically, the dexamethasone microspheres of Example 10 were administered to rats, and the changes in the blood concentration of dexamethasone (free base) over time were measured and shown in Table 8 and FIG. 2. The drug microspheres used in FIG. 2 show the results of Example 10. The following Table shows the cumulative AUC (%) values over time, where h in the elapsed time represents hour and d represents day.

TABLE 8

| | |
|---|---|
| Elapsed time | Example 10 |

| | | |
|---|---|---|
| 1 | h | 0.1 |
| 24 | h | 3 |
| 7 | d | 15.7 |
| 28 | d | 43.3 |
| 56 | d | 63.5 |
| 84 | d | 77.4 |
| 112 | d | 89.9 |
| 140 | d | 100 |

Referring to Table 8, the microspheres of Example 10 showed continuous release for up to about 140 days, representing that it was a formulation suitable for a long-term release of drug.

The drug microspheres of Examples 23, 24 and 25 were suspended at an amount of drug microspheres corresponding to a dose of 0.3 mg/head as dexamethasone acetate in the microspheres manufactured with the theoretical content of 60% of the used drug, and then were injected subcutaneously into SD rats. Blood was collected every hour, and the blood dexamethasone concentration was measured using LC-MS/MS.

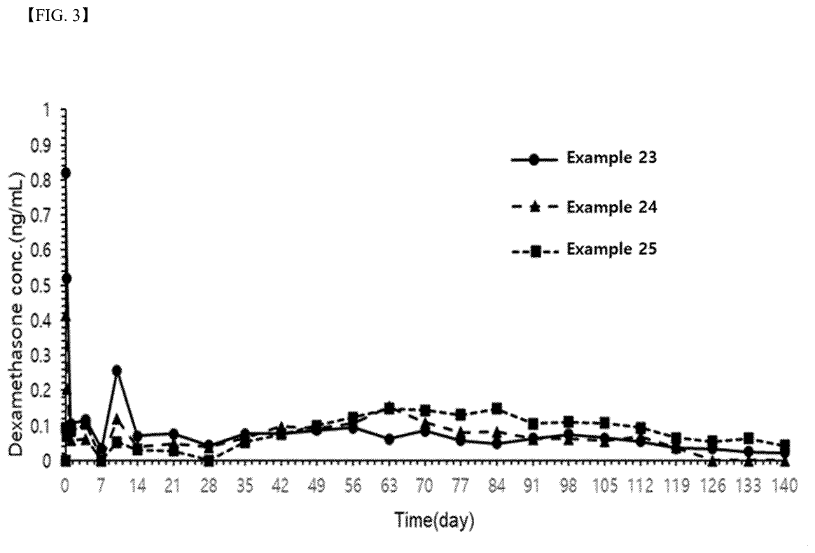

Specifically, the dexamethasone microspheres of Examples 23, 24, and 25 were administered to rats, and the changes in the blood concentration of dexamethasone (free base) over time were measured and shown in Table 9 and FIG. 2. The drug microspheres used in FIG. 3 show the results of Examples 23, 24 and 25 The following Table shows the cumulative AUC (%) values over time, where h in the elapsed time represents hour and d represents day.

TABLE 9

| | | | |
|---|---|---|---|
| elapsed time | Example 23 | Example 24 | Example 25 |

| | | | | |
|---|---|---|---|---|
| 1 | h | 0.2 | 0.1 | 0 |
| 24 | h | 3.7 | 1.8 | 0.7 |
| 7 | d | 9.3 | 3.8 | 3.1 |
| 28 | d | 29.5 | 15.9 | 7 |
| 56 | d | 51.9 | 41.6 | 24.5 |
| 84 | d | 71.8 | 75.9 | 58.3 |
| 112 | d | 90.3 | 95.9 | 85 |
| 140 | d | 100 | 100 | 100 |

Examples 23, 24, and 25 in Table 9 are microspheres manufactured with a theoretical drug content (amount of drug used) of 60% or more. As can be seen from the results in Table 9, drug microsphere formulations with a drug content of 60% or more also exhibit a steady release profile for more than 84 days.

The drug microspheres of Examples 13, 19, and 27-29 were suspended at an amount of drug microspheres corresponding to a dose of 0.3 mg/head as dexamethasone acetate in the microspheres, and then were injected subcutaneously into SD rats. Blood was collected every hour, and the blood dexamethasone concentration was measured using LC-MS/MS.

Specifically, the dexamethasone microspheres of Examples 13, 19, and 27-29 were administered to rats, and the changes in the blood concentration of dexamethasone (free base) over time were measured and shown in Table 10. Table 10 below shows the cumulative AUC (%) values over time, where h in the elapsed time represents hour, d represents day, and (nd) represents no measured value due to the end of the test period.

TABLE 10

| | | Exam- | Exam- | Exam- | Exam- | Exam- |
|---|---|---|---|---|---|---|
| elapsed time | | ple 13 | ple 19 | ple 27 | ple 28 | ple 29 |
| 1 | h | 0.01 | 0.02 | 0.29 | 0.16 | 0.07 |
| 24 | h | 0.18 | 0.51 | 5.27 | 2.98 | 1.4 |
| 7 | d | 1.23 | 1.72 | 17.93 | 10.41 | 4.87 |
| 28 | d | 5.31 | 5.11 | 50.23 | 30.02 | 14.18 |
| 56 | d | 11.04 | 9.41 | 70.14 | 43.55 | 21.87 |
| 84 | d | 19.61 | 15.01 | 84.5 | 55.31 | 29.79 |
| 112 | d | 46.44 | 21.39 | 95.04 | 73.17 | 40.9 |
| 140 | d | 88.9 | 29.91 | 98.73 | 94.31 | 54.93 |
| 168 | d | 98.94 | 42.18 | 100 | 99.52 | 64.58 |
| 196 | d | 100 | 63.82 | nd | 100 | 77.96 |
| 224 | d | nd | 85.47 | nd | nd | 91.15 |
| 294 | d | nd | 100 | nd | nd | 100 |

Each microspheres of Examples 13, 19, and 27 in Table 10 had a release period of 84 days or longer, but these microspheres represented the phenomenon that AUC was insufficient or the release period was shortened during a specific period. In order to complement these points, each microsphere was mixed at a specific ratio to complement the deficiencies of each formulation, so as to produce a formulation with a steady release profile for more than 84 days from immediately after administration.

### Test Example 6. Analysis of the Cross-Section of Drug Microspheres

This experiment was conducted to analyze the cross-section of microspheres manufactured according to the above Examples and Comparative Examples, and to test the effects of co-solvent and amount of used drug (target loading) on the cross-section of microspheres.

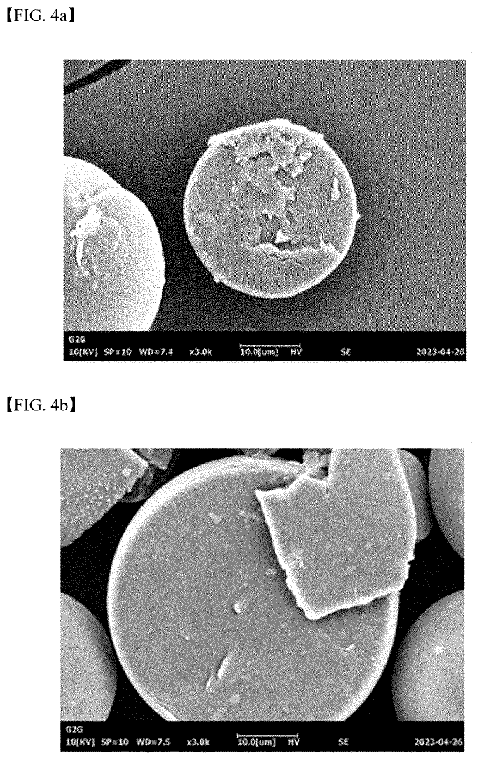

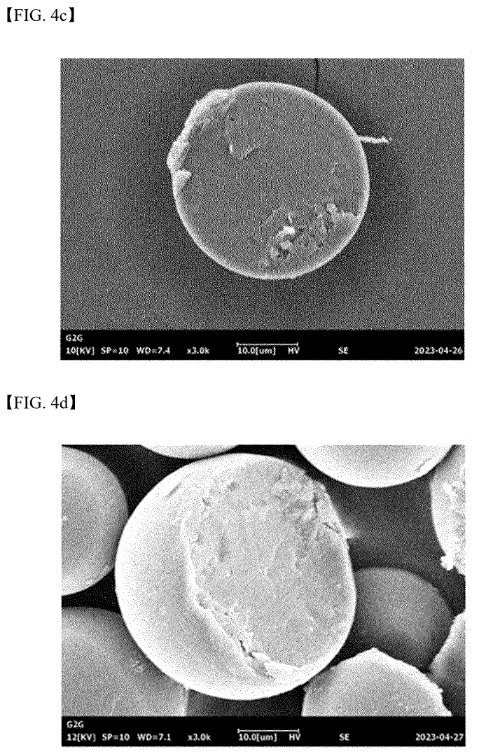

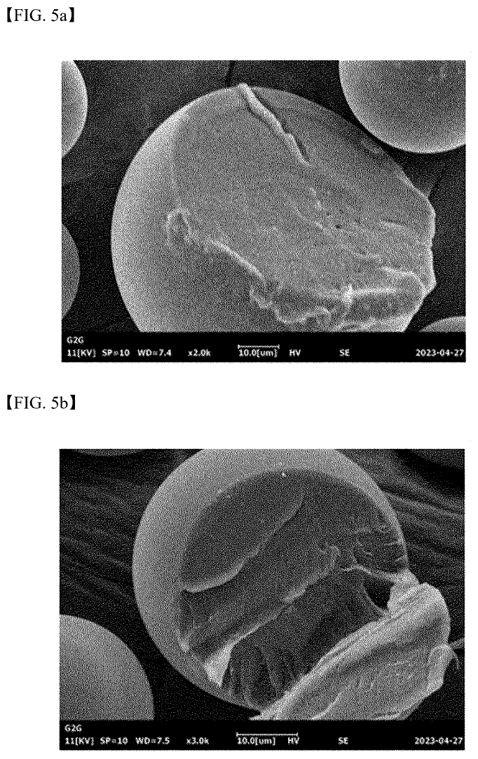

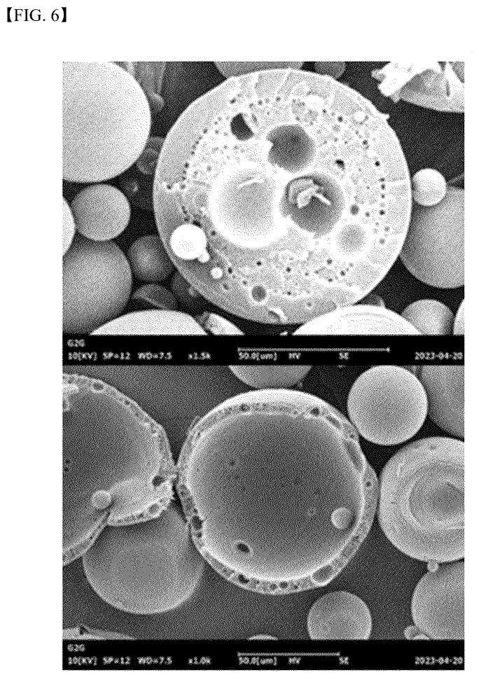

Specifically, the cross-sectional analysis of the microspheres was performed by repeatedly cutting the cross-sections of the microspheres using a square cross-section knife several times, and observing the cross-sections of the microspheres using a scanning electron microscope (SEM). FIG. 4a to FIG. 4d show the analysis of the microspheres according to the TL (Target Loading) of drug microspheres manufactured using benzyl alcohol as a co-solvent where TL was divided into 40%, 50%, 60%, and 70%. FIG. 5 shows the analysis of the microspheres according to the TL (Target Loading) of drug microspheres manufactured using DMF as a co-solvent where TL was divided into 20% and 40%. FIG. 6 shows the analysis of the microspheres manufactured using DMSO as a co-solvent (Comparative Example 1).

As a result, the results of observing the cross-sections of the microparticles of Examples 5, 16, 23, and 26 with a scanning electron microscope (SEM) are shown in FIGS. 4a to 4d, and the results of observing the cross-sections of the microparticles of Examples 6 and 7 with a scanning electron microscope (SEM) are shown in FIGS. 5a and 5b. In addition, the results of observing the cross-sections of the microparticles of Comparative Example 1 with a scanning electron microscope (SEM) are shown in FIG. 6.

As shown in the experimental results, the microspheres according to the theoretical drug content had a clean outer surface, showed a dense cross-section without internal pores, and exhibited excellent encapsulation efficiency regardless of the theoretical drug content. The drug microspheres manufactured using benzyl alcohol or DMF as a co-solvent, showed a dense cross-section with almost no pores formed inside the microspheres, whereas the microspheres manufactured using DMSO as a co-solvent had many pores formed inside the microspheres and had not dense cross-section.

### Test Example 7. Measurement of Porosity within Drug Microspheres

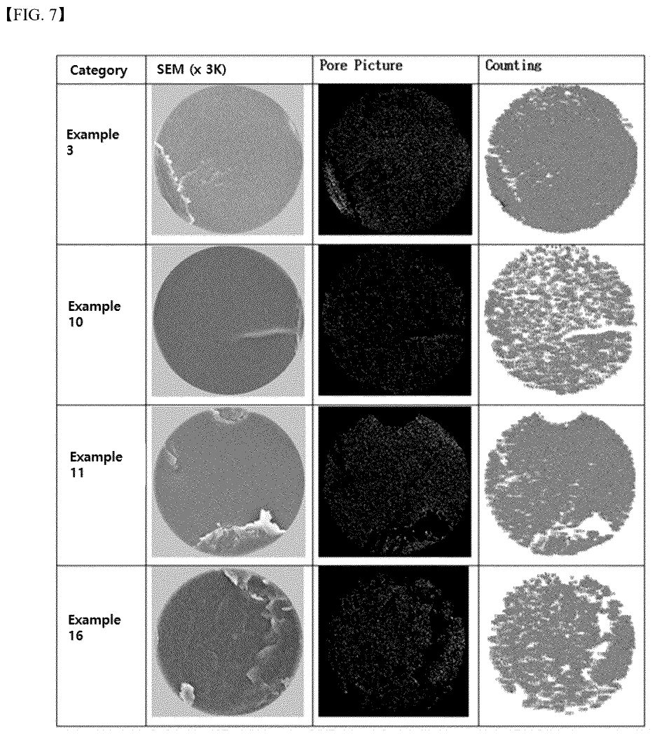

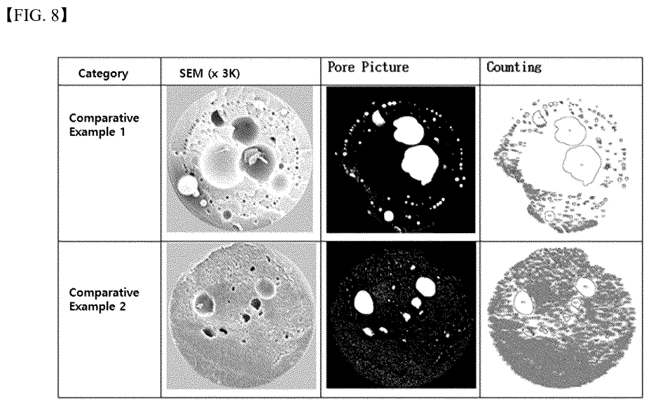

This experiment was conducted to analyze the cross-section of microspheres manufactured according to the Examples and Comparative Examples, and then to test the effects of the co-solvent and the amount of drug used (target loading) on the internal porosity of the microspheres. Specifically, the cross-sectional analysis of the microspheres was performed by repeatedly cutting the cross-sections of the microspheres using a square cross-section knife several times, and observing the cross-sections of the microspheres using a scanning electron microscope (SEM). Then, the diameter and porosity of the pores inside the microspheres were measured using the Image J program. FIG. 7a to FIG. 7b show the analysis of the microspheres according to the TL (Target Loading) of drug microspheres manufactured using benzyl alcohol and dimethyl sulfoxide as a co-solvent where TL was divided into 20%, 40%, and 50%.

As a result, the results of observing the cross-sections of the microspheres of Examples 3, 10, 11, and 16 are shown in FIG. 7 and Table 11, and the results of observing the cross-sections of the microspheres of Comparative Examples 1 and 2 are shown in FIG. 8 and Table 11.

As shown in the experimental results, the microspheres prepared by using benzyl alcohol had a porosity of the internal pores of less than 5%, and the microspheres prepared by using dimethyl sulfoxide had a porosity of the internal pores of up to 13%. The more internal pores there are, the higher the possibility that sustained release will not occur after microsphere administration and a burst of drug may occur during the release period. Thus, the relatively smaller internal pores there are, the more stable the release can be induced.

TABLE 11

| | | | Average | Maximum |
|---|---|---|---|---|
| | Porosity | Average area of | diameter of | diameter of |
| Category | (%) | pore (μm²) | pore(μm) | pore(μm) |
| Example 3 | 4.81 | 0.013 | 0.12 | 0.82 |
| Example 10 | 1.87 | 0.010 | 0.11 | 0.32 |
| Example 11 | 4.50 | 0.013 | 0.12 | 0.58 |
| Example 16 | 2.98 | 0.012 | 0.12 | 0.44 |
| Comparative | 13.44 | 2.528 | 0.59 | 18.45 |
| Example 1 | | | | |
| Comparative | 8.51 | 0.198 | 0.31 | 8.57 |
| Example 2 | | | | |

1. A sustained-release injectable preparation comprising a drug microsphere containing a drug and a biocompatible polymer,
wherein the drug is dexamethasone acetate, and a content of the drug is 15 to 70 wt % based on 100 wt % of the drug microsphere.

2. The sustained-release injectable preparation according to claim 1, wherein a circularity span value of the drug microparticles is from 0.01 to 0.05.

3. The sustained-release injectable preparation according to claim 2, wherein an average circularity of the drug microparticles is 0.87 to 1.00.

4. The sustained-release injectable preparation according to claim 1, wherein a porosity of the drug microspheres is 8% or less.

5. The sustained-release injectable preparation according to claim 1, wherein a maximum diameter of the pores in the drug microspheres is 8 micrometers (μm) or less.

6. The sustained-release injectable preparation according to claim 1, wherein the maximum diameter of the pores in the drug microspheres is 8 micrometers (μm) or less and an average diameter is 0.3 micrometers (μm) or less.

7. The sustained-release injectable preparation according to claim 1, wherein a span value of diameter of the drug microspheres is less than 1.1.

8. The sustained-release injectable preparation according to claim 1, wherein the dexamethasone acetate is at least one selected from the group consisting of dexamethasone 17-acetate and dexamethasone 21-acetate.

9. The sustained-release injectable preparation according to claim 1, wherein the drug microspheres have a drug-release property in which an amount of dexamethasone released for 24 hours is 15% or less based on 100% of the drug contained in the microspheres in an in-vitro drug release test using a phosphate buffer (pH 7.4).

10. The sustained-release injectable preparation according to claim 1, wherein the drug microspheres have a drug-release property in which a cumulative drug release amount (AUC) is 15% or less for 24 hours when administered intramuscularly to SD rats.

11. The sustained-release injectable preparation according to claim 1, wherein the drug microspheres have an average particle diameter of 10 to 100 μm.

12. The sustained-release injectable preparation according to claim 1, wherein the sustained-release injectable preparation comprises two or more types of drug microspheres having different compositions.

13. The sustained-release injectable preparation according to claim 12, wherein the two or more types of drug microspheres having different compositions have different polymer types and drug contents of the microspheres.

14. The sustained-release injectable preparation according to claim 13, wherein the drug microspheres having different polymer types are at least one selected from the group consisting of polymers having different repeating units, polymers having the same repeating unit but having different terminal groups, and polymers having different intrinsic viscosities.

15. The sustained-release injectable preparation according to claim 1, wherein the drug microspheres are used for arthritis, Meniere's disease, macular degeneration or solid tumors.

16. A method for producing drug microspheres comprising steps of:
(a) dissolving a biodegradable polymer and a drug in an organic solvent to form a dispersed phase solution;
(b) homogeneously mixing the biodegradable polymer solution prepared in step (a) in an aqueous solution containing a surfactant to form an emulsion including a dispersed phase solution and an aqueous solution containing the surfactant as a continuous phase;
(c) extracting an organic solvent from the dispersed phase of the emulsion prepared in step (b) toward the continuous phase, and evaporating the organic solvent to prepare microspheres; and
(d) recovering the microspheres from the emulsion of step (c),
wherein a co-solvent is used with the organic solvent in step (a).

17. The method of claim 16, wherein the co-solvent is at least one selected from the group consisting of benzyl alcohol and dimethylformamide.

18. The method of claim 16, wherein the drug microspheres have one or more properties of:
1.2 or less of a diameter span value of the drug microspheres,
0.87 to 1.00 of an average circularity of the drug microspheres,
8% or less of a porosity of the drug microspheres,
8 micrometer (μm) or less of a maximum diameter of pores in the drug microspheres, and
0.3 micrometer (μm) or less of an average diameter of pores in the drug microspheres.

19. The method of claim 16, further comprising a step of controlling a release property of the microspheres by adjusting a temperature of the continuous phase at a glass transition temperature of the biocompatible polymer or higher in the step (c).

20. The method of claim 16, wherein the step of controlling a release property of the microspheres is performed by adjusting a temperature of the continuous phase within a temperature range from a glass transition temperature (Tg) of the polymer as a lower limit to (Tg+30° C.) as a upper limit.
